(19) United States
(12) Patent Application Publication
(10) Pub. No.: US 2010/0158876 A1
(43) Pub. Date: Jun. 24, 2010

(54) PROCESS FOR THE PREPARATION OF STEM CELLS FROM HUMAN MUSCLE TISSUE AND ADIPOSE TISSUE, AND STEM CELLS OBTAINABLE BY THIS PROCESS

(76) Inventors: Giulio ALESSANDRI, Milano (IT); Arnaldo Caruso, Brescia (IT); Josè Sebastian Franzone, Torino (IT)

Correspondence Address: Steinfl & Bruno, 301 N Lake Ave Ste 810, Pasadena, CA, 91101, US

(21) Appl. No.: 12/607,934

(22) Filed: Oct. 28, 2009

Related U.S. Application Data
(63) Continuation of application No. 10/510,622, filed on Jun. 27, 2005, now abandoned.

(30) Foreign Application Priority Data
Apr. 10, 2002 (IT) TO2002A000311

(51) Int. Cl. A61K 35/34; C12N 5/071

(52) U.S. Cl. 424/93.7; 435/366; 435/325

(57) ABSTRACT

The invention relates to a process for the preparation of the human stem cells from muscle tissue or adipose tissue. The process provides for the incubation of cells obtained from a sample of muscle tissue or adipose tissue in a medium comprising BSA, Bfgf, EGF, VEGF, LIF, heparin and usual inorganic salts, natural acids and vitamins necessary for the growth of mamalian cells. The invention relates also to the human muscle stem cells (hMSC) and human adipose tissue cells (hFSC) obtainable by this process.

The present invention relates to a process for the preparation of human stem cells from a sample of human adipose or muscle tissue, as well as human stem cells obtainable by this process.

In particular, the invention relates to the preparation of human stem cells of the muscle (hMSC) and of the adipose tissue (hFSC) from a sample of skeletal muscle tissue and of adipose tissue, respectively.

It is known that skeletal muscle has a regenerative capacity owing to the presence of immature progenitor muscle cells. All muscle fibres in fact contain cells capable of growing and differentiating to form muscle fibres; these cells are called satellite cells. Satellite cells are generally dormant and remain in a non-differentiated form under the basal lamina of the muscle fibre. A lesion of the muscle activates these cells, bringing them from the dormant phase into the growth phase. Some of these cells differentiate to form myocytes which, by fusing with one another, lead to the regeneration of a new muscle fibre, thus restoring normal muscle function. Another portion of the satellite cells remain in a non-differentiated form, returning the number of satellite cells in the muscle fibre to the original amount.

The present inventors have now developed a process which permits the production, starting from a sample of human muscle tissue, of stem cells which are even more non-differentiated than satellite cells, because they are capable of differentiating both to form satellite cells and also to form various cell elements, such as nerve cells (neurones, gliocytes, astrocytes), vascular cells (endothelium) and bone cells (osteoblasts).

The present inventors have also used the same preparation process on samples of adipose tissue and this has enabled them to obtain adipose tissue stem cells that are likewise capable of differentiating both to form muscle cells (smooth and striated) and to form nerve cells (neurones, gliocytes, astrocytes), vascular cells (endothelium) and bone cells (osteoblasts).

The present invention therefore relates firstly to a process for the preparation of human stem cells from a sample of human adipose or muscle tissue, comprising the steps of:
a) preparing a cell suspension from a sample of human adipose or muscle tissue;
b) recovering the cells from the cell suspension; and
c) incubating these cells in a medium comprising BSA, bFGF, EGF, VEGF, LIF, heparin and usual inorganic salts, natural amino acids and vitamins necessary for the growth of mammalian cells.

The medium used for the incubation of the cells is preferably the medium DMEM/F12 supplemented with from 0.4% to 0.8% of BSA, from 5 to 20 ng/ml of bFGF, from 10 to 40 ng/ml of EGF, from 2.5 to 10 ng/ml of VEGF, from 5 to 20 ng/ml of LIF, from 1 to 20 μg/ml of heparin, from 1.8 to 3 mg/ml of glucose, from 2 to 2.5 mg/ml of NaHCO₃, from 2.5×10⁻³ to 7.5×10⁻³ M of Hepes, from 50 to 200 μg/ml of apotransferrin, from 10 to 30 μg/ml of insulin, from 3×10⁻⁴ to 7×10⁻⁴ M of putrescine, from 4×10⁻⁸ to 8×10⁻⁸ M of selenium, from 1×10⁻⁸ to 3×10⁻⁸ M of progesterone.

The easy accessibility of samples of muscle and of adipose tissue makes these tissues an ideal source for the isolation and growth of stem cells.

The present invention relates also to a human muscle stem cell (hMSC) obtainable by means of the process described above in which a sample of human skeletal muscle tissue is used as the starting tissue.

Yet another subject of the present invention is a human adipose tissue stem cell (hFSC) obtainable by means of the process described above in which a sample of human adipose tissue is used as the starting tissue.

In order to obtain hMSC cells, step a) of the process according to the invention preferably comprises the digestion of the sample of human skeletal muscle tissue with trypsin.

In addition, according to a preferred embodiment for obtaining hMSC cells, the incubation step c) comprises:
c₁) resuspending the cells recovered from the cell suspension of step a) in the growth medium defined above;
c₂) incubating the cell suspension obtained in the previous step inside a container for cell cultures, which has previously been treated with type I collagen, for from 18 to 24 hours at a temperature of approximately 37° C. and in a 5% CO₂ atmosphere;
c₃) removing the growth medium from the container and replacing it with an identical freshly prepared growth medium; and
c₄) incubating for a further 48 to 72 hours, thereby obtaining the formation of small roundish cells adhering to the walls of the container, the small adherent roundish cells being human muscle stem cells (hMSC).

Alternatively, according to a preferred embodiment for obtaining hFSC cells, the incubation step c) comprises:
c₁) resuspending the cells recovered from the cell suspension of step a) in the growth medium defined above;
c₂) incubating the cell suspension obtained in the previous step inside a container for cell cultures, which has previously been treated with type I collagen, for from 18 to 24 hours at a temperature of approximately 37° C. and in a 5% CO₂ atmosphere;
c₃) recovering the cells not adhering to the walls of the container and resuspending them in freshly prepared growth medium as defined above;
c₄) placing the cell suspension obtained in the previous step in a second container for cell cultures which has not previously been treated with collagen and cultivating the cells therein for from 7 to 10 days, thereby obtaining the formation of floating cell aggregates, the cells of these aggregates being human adipose tissue stem cells (hFSC).

In this embodiment, steps c₂) and c₃) are preferably repeated twice more—for a total of 3 changes of medium—before proceeding to the subsequent step c₄) of incubation in the container not treated with collagen.

Owing to their capacity to differentiate to form a multiplicity of various cell types, the stem cells hMSC and hFSC of the, present invention can be used in a variety of therapeutic applications, such as:
the treatment of ischaemic tissue after thrombotic or traumatic phenomena or the repair of vascular damage caused by traumatic phenomena or of atherosclerotic origin (for these applications the stem cells can be engineered by the introduction into their genome of angiogenic factors, such as, for example, VEGF (“vascular endothelial growth factor”));
the regeneration of striated muscle tissue;
the regeneration of skeletal muscle tissue owing to traumatic events;
the cell treatment of myocardial infarct;
the regeneration of bone tissue and cartilaginous tissue;
in co-transplantation with other stem cells, such as, for example, medullary or neuronal cells, for supporting establishment and growth and promoting the regeneration of mesenchymal tissue (bone, cartilage, smooth and vascular muscle);
in the establishment of grafted bone tissue and, in general, in all conditions that require the establishment and growth of cells and tissue in the human organism;
the production of growth and/or trophic factors for cells of various origins and sources;
the production of hormones for therapeutic purposes in humans;
tissue bioengineering;
the regeneration of peripheral nerves;
the treatment of multiple sclerosis;
the regeneration of central nervous tissue;
the treatment of Parkinson's disease and Alzheimer's disease.
   Isolation of hMSC

A bioptic sample of human skeletal muscle, after being weighed and preferably catalogued and recorded, is transferred into a culture petri dish and broken up finely with a bistoury into fragments of approximately 1 mm³ or less.

After the addition of PBS (isotonic phosphate buffer) and antibiotics, the fragments are transferred into a conical test tube and washed 3 times with PBS by light centrifuging at 200 rpm at 4° C. When the final washing operation has been completed and the supernatant has been discarded, a solution of 0.25% (weight/volume) trypsin and 0.25% EDTA (ethylenediaminetetraacetic acid) is added to the fragments. The volume quantity of trypsin to be added is calculated relative to the volume of fragmented tissue: for approximately 0.5 ml of tissue, approximately 3 ml of enzyme solution are added. The test tube is then transferred to a bath maintained at a constant temperature of 37° C. and is incubated for approximately 2 hours with slight agitation.

When the incubation of the fragments with trypsin is complete, the test tube is left to stand for approximately 10 minutes at ambient temperature in order to cause all of the undigested material to settle at the bottom of the test tube. The cells in suspension are sucked up with a Pasteur pipette and transferred into a fresh test tube containing the same volume of DMEM medium to which 10% FCS (foetal calf serum) has been added, this being used to block the action of the trypsin. The cells are then recovered by centrifuging at 1000 rpm for 10 minutes. The pellet obtained is subsequently washed 3 times with PBS by centrifuging in order to remove all of the FCS. Finally, the cell pellet obtained is resuspended in a growth medium for stem cells called HUMAN-G which is composed of DMEM/F12 medium (Gibco) containing: 0.8% of BSA (Bovine Serum Albumin), 10 ng/ml of bFGF (basic Fibroblast Growth Factor), 20 ng/ml of EGF (Epidermal Growth Factor), 5 ng/ml of IMF (Vascular Endothelial Growth Factor), 10 ng/ml of LIF (Lymphocyte Inhibitor Factor), 10 μg/ml of heparin, 2.4 mg/ml of glucose, 2.25 mg/ml of NaHCO₃, 5×10⁻³ M of Hepes, 100 μg/ml of apotransferrin, 25 μg/ml of insulin, 6×10⁻⁴ M of putrescine, 6×10⁸ M of selenium, and 2×10⁻⁸ M of progesterone.

The cells are then sown in a T 25 culture flask previously treated with type I collagen in order to promote cell adhesion. The flask is then incubated for 18-24 hours in an incubator at 37° C. with 5% of CO₂. At the end of the incubation period, the medium is removed and replaced with an identical freshly prepared HUMAN-G medium; the flask is then returned to the incubator for a further 48-72 hours.

The adhering cells in the culture flask are initially composed of a population of small spindle-shaped cells (that is to say, the satellite cells of the striated muscle), while the cells in suspension in the medium are generally red corpuscles or dead cells and are readily removed by suction. After approximately 48-72 hours' incubation, small roundish cells, that is to say, the muscle stem cells (hMSC), start to appear at the bottom of the culture together with the satellite cells.

These small roundish cells occur only when using the culture medium described above. In fact, with other growth media known in the art these small roundish cells do not develop.

After approximately 1-2 weeks from the initial sowing, the presence of cells in suspension, in addition to the spindle-shaped satellite cells and the small roundish cells, starts to be observed. These cells are not capable of multiplying autonomously but their number nevertheless increases as the culture progresses, suggesting that they originate from the small roundish cells. Therefore the cells in suspension do not represent another cell population but probably an intermediate stage of differentiation of the adhering stem cells. After approximately one month's culture, it is possible to obtain muscle stem cells (hMSC) in a reasonable amount. Under these experimental conditions, the hMSC can multiply in culture and can reach reasonable numbers (2-3×10⁶) without showing any particular signs of morphological variation at least after 3 months' culture. After this period, growth decreases and cells having the very elongate morphology characteristic of differentiated muscle cells start to appear. In addition, if the cells in suspension are removed from the hMSC culture and are cultivated on proteins of the basal membrane, such as laminin, they are able to differentiate to form cells of nervous origin (astrocytes, neurones) that is to say, differing from the original muscle tissue.

Isolation of hFSC

A sample of adipose tissue is weighed, transferred to a culture plate and washed with large amounts of PBS. After this operation, the adipose tissue, being generally very loose, does not require the mechanical breaking-down with bistouries which is necessary for muscle tissue. It is therefore readily broken down by the mechanical action of resuspension with a Pasteur pipette. The tissue is subsequently washed with PBS and transferred into a test tube and left to stand at ambient temperature for approximately 10 minutes. This procedure enables all of the floating fatty tissue to rise to the surface, while the connective component settles at the bottom of the test tube. The fatty component is then recovered with a pipette and transferred to a fresh test tube containing a 0.25% collagenase solution. The amount of enzyme solution to be added to the adipose tissue depends on the amount of material to be processed: for approximately 1 ml of fat, approximately 2 ml of enzyme solution are added. The material is incubated for approximately 2 hours at 37° C. with slight agitation, after which the digested tissue is washed 2 or 3 times with PBS by centrifuging at 1000 rpm for 10 minutes. The cell pellet obtained is resuspended in PBS and the cell suspension obtained is filtered (filter having a porosity of 30 μm) in order to remove all of the vascular fragments which are present in large amounts in the adipose tissue and which often have dimensions larger than 30 μm. All of the cells or the cell microaggregates having dimensions smaller than 30 μm are recovered by centrifuging (1000 rpm for 10 minutes), the supernatant is discarded and the cell pellet is resuspended in HUMAN-G medium as described above with regard to the production of hMSC, with the only variant that the concentrations of LIF and VEGF are 20 ng/ml and 10 ng/ml, respectively. The cells are then sown in a T 25 flask treated with collagen and incubated at 37° C. in a 5% CO₂ atmosphere for approximately 18-24 hours. The differentiated cells, the vascular fragments and the fibroblasts present in the preparation adhere to the culture flask, while all of the dead cells and the non-differentiated cells (that is to say, stem cells) continue to float in the medium. This procedure is generally repeated 3 times in order to be reasonably sure that all of the differentiated cells have been removed from the preparation. At the end of the final removal operation, the non-adhering cells are centrifuged at 500 rpm, resuspended in fresh HUMAN-G and sown in T 25 flasks not treated with collagen. After approximately 7-10 days from initial culturing, formations of several aggregate cells which float in the culture medium are observed. These cells are hFSC. After approximately 30-45 days' culture, the amount of stem cells reaches a reasonable number (approximately 2-3×10⁶).

Phenotypic and Functional Characterisation of hMSC

An analysis was carried out in respect of the expression of various markers on the hMSC cells according to the invention.

By using techniques of cytofluorimetry, the positivity of the hMSC cells to CD34 and Bcl-2+, which are markers for which murine MSC were found to be positive in previous studies, was confirmed. Positivity to KDR/Flk-1 and Sca-1, that is to say, markers that are also found on bone marrow stem cells, was also confirmed.

The expression of muscle markers, such as desmine and myogenin, was evaluated by immunohistochemical and immunofluorescence studies.

In order to evaluate the characteristics of differentiation of hMSC to form cells of nervous origin, the neuronal phenotypic markers were analyzed at various culture times. The cells were cultivated for from 7 to 24 days on a substrate of laminin in the presence of culture medium without growth factors. After approximately 7-10 days, the presence of GAD, a marker for GABAergic neurones, was detected, which indicates differentiation to form neurones of the peripheral nervous system. It was also observed that hMSC was positive to GFAP, which suggests differentiation to form cells of the glia (gliocytes).

After approximately 21 days, an analysis was carried out in respect of the presence of neurofilaments-M (NFM), which was found to be positive only to a limited extent.

The differentiation of the hMSC cells to form smooth and striated muscle cells was analyzed using desmine antibodies. For that purpose, the cells were cultivated on collagen substrates in medium without growth factors in the presence of 3% FCS.

Differentiation to form bone cells (osteoblasts) was analyzed by assessing the presence of osteocalcin, a protein which is specifically produced by osteoblasts.

Because the literature has reported the existence of a progenitor common to the endothelial cells and the muscle cells of vessels, the observed capacity of hMSC to differentiate to form muscle tissue cells would suggest that these cells are also capable of differentiating to form endothelial phenotype.

Because the hFSC of the present invention are of the same mesenchymal origin as the hMSC, this also suggests that the same differentiating abilities described above contained in the hMSC are also present in the hFSC.

1-14. (canceled)

15. A process for preparation of human stem cells from a sample of human adipose or muscle tissue, said human stem cells being capable of differentiating into nerve cells, vascular cells and bone cells, the process comprising:
a) preparing a cell suspension from a sample of human adipose or muscle tissue;
b) recovering cells from the cell suspension; and
c) incubating the cells in a medium comprising BSA, bFGF, EGF, VEGF, LIF, heparin and usual inorganic salts, natural amino acids and vitamins.

16. The process according to claim 15, wherein the medium is DMEM/F12 and comprises: from 0.4% to 0.8% of BSA, from 5 to 20 ng/ml of bFGF, from 10 to 40 ng/ml of EGF, from 2.5 to 10 ng/ml of VEGF, from 5 to 20 ng/ml of LIF, from 1 to 20 μg/ml of heparin, from 1.8 to 3 mg/ml of glucose, from 2 to 2.5 mg/ml of NaHCO3, from 2.5×10⁻³ to 7.5×10⁻³ M of Hepes, from 50 to 200 μg/ml of apotransferrin, from 10 to 30 μg/ml of insulin, from 3×10⁻⁴ to 7×10⁻⁴ M of putrescine, from 4×10⁻⁸ to 8×10⁻⁸ M of selenium, and from 1×10⁻⁸ to 3×10 ⁻⁸ M of progesterone.

17. The process according to claim 15, wherein the tissue sample is a human skeletal muscle sample and the stem cells are human muscle stem cells.

18. The process according to claim 17, wherein step a) comprises digestion of the human skeletal muscle sample with trypsin.

19. The process according to claim 17, wherein step c) comprises:
resuspending cells recovered from the cell suspension of step a) in the medium thus obtaining a recovered cell suspension;
incubating the recovered cell suspension inside a container for cell culture previously treated with type I collagen, for from 18 to 24 hours at a temperature of approximately 37° C. and in a 5% CO₂ atmosphere;
removing the medium from the container and replacing, the removed medium with a freshly prepared medium comprising BSA, bFGF, EGF, VEGF, LIF, heparin and usual inorganic salts, natural amino acids and vitamins; and
incubating for a further 48 to 72 hours, thereby obtaining formation of small roundish cells adhering to the walls of the container, the adhering small roundish cells being human muscle stem cells.

20. Human muscle stem cells capable of differentiating into nerve cells, vascular cells and bone cells, the human muscle stem cells obtained by the process according to claim 17.

21. The process according to claim 15, wherein the tissue sample is a human adipose tissue sample and the stem cells are human adipose tissue stem cells.

22. The process according to claim 21, wherein step c) comprises:
resuspending the cells recovered from the cell suspension of step a) in the medium thus obtaining a recovered cell suspension;
incubating the recovered cell suspension inside a first container for cell cultures, the first container previously treated with type I collagen, for from 18 to 24 hours at a temperature of approximately 37° C. and in a 5% CO₂ atmosphere;
recovering cells not adhering to the walls of the container and resuspending the recovered not adhering cells in freshly prepared medium comprising BSA, bFGF, EGF, VEGF, LIF, heparin and usual inorganic salts, natural amino acids and vitamins, thus obtaining a recovered not adhering cell suspension; and
placing the recovered not adhering cell suspension in a second container for cell cultures, the second container not previously treated with collagen and cultivating the cells therein for from 7 to 10 days, thus obtaining the formation of floating cell aggregates, cells of these aggregates being human adipose tissue stem cells.

23. The process according to claim 22, wherein the incubating and the recovering are repeated twice more before the placing.

24. Human adipose tissue stem cells capable of differentiating into nerve cells, vascular cells and bone cells, the human adipose tissue stem cells obtained by the process according to claim 21.

25. A method of using the stem cells according to claim 20, the method comprising:
using the stem cells for regenerating of a tissue selected from the group consisting of bone tissue, cartilaginous tissue, endothelial tissue, smooth muscle tissue, striated muscle tissue and nerve tissue.

26. A method of using the stem cells according to claim 20, the method comprising:
using the stem cells for one or more of: treatment of an ischaemic tissue, repair of vascular damage, cell treatment of myocardial infarct, co-transplantation with other stem cells or tissues, production of growth and/or trophic factors, production of hormones, tissue bioengineering, regeneration of peripheral nerves, treatment of multiple sclerosis, treatment of myocardial infarct, treatment of Alzheimer's disease and treatment of Parkinson's disease.

27. A medicament for regeneration of a tissue selected from the group consisting of bone tissue, cartilaginous tissue, endothelial tissue, smooth muscle tissue, striated muscle tissue and nerve tissue, the medicament comprising the human muscle stem cells according to claim 20.

28. A medicament for treatment of an ischaemic tissue, repair of a vascular damage, cell treatment of myocardial infarct, co-transplantation with other stem cells or tissues, regeneration of peripheral nerves, treatment of multiple sclerosis, treatment of myocardial infarct, treatment of Alzheimer's disease or treatment of Parkinson's disease, the medicament comprising the human muscle stem cells according to claim 20.

29. A method of using the stem cells according to claim 24, the method comprising:
using the stem cells for regenerating a tissue selected from the group consisting of bone tissue, cartilaginous tissue, endothelial tissue, smooth muscle tissue, striated muscle tissue and nerve tissue.

30. A method of using the stem cells according to claim 24, the method comprising:
using the stem cells for one or more of: treatment of an ischaemic tissue, repair of vascular damage, cell treatment of myocardial infarct, co-transplantation with other stem cells or tissues, production of growth and/or trophic factors, production of hormones, tissue bioengineering, regeneration of peripheral nerves, treatment of multiple sclerosis, treatment of myocardial infarct, treatment of Alzheimer's disease and treatment of Parkinson's disease.

31. A medicament for regeneration of a tissue selected from the group consisting of bone tissue, cartilaginous tissue, endothelial tissue, smooth muscle tissue, striated muscle tissue and nerve tissue, the medicament comprising the human muscle stem cells according to claim 24.

32. A medicament for treatment of an ischaemic tissue, repair of a vascular damage, cell treatment of myocardial infarct, co-transplantation with other stem cells or tissues, regeneration of peripheral nerves, treatment of multiple sclerosis, treatment of myocardial infarct, treatment of Alzheimer's disease or treatment of Parkinson's disease, the medicament comprising the human muscle stem cells according to claim 24.
